(19) United States
(12) Patent Application Publication
(10) Pub. No.: US 2004/0002447 A1
(43) Pub. Date: Jan. 1, 2004

(54) Induction of insulin expression

(75) Inventors: Fred Levine, Del Mar, CA (US); Pamela Itkin-Ansari, Carlsbad, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: REGENTS OF THE UNIVERSITY OF CALIFORNIA, Oakland, CA

(21) Appl. No.: 10/162,952

(22) Filed: Jun. 4, 2002

(51) Int. Cl. A61K 38/00

(52) U.S. Cl. 514/9

(57) ABSTRACT

The present invention provides compositions and methods for inducing insulin expression in cells by contacting the cells with a histone deacetylase inhibitor.

### STATEMENT AS TO RIGHTS TO INVENTIONS MADE UNDER FEDERALLY SPONSORED RESEARCH OR DEVELOPMENT

This invention was made with Government support under Grant No. DK55283, awarded by the National Institutes of Health. The Government has certain rights in this invention.

### BACKGROUND OF THE INVENTION

Transplantation of cells exhibiting glucose-responsive insulin secretion has the potential to cure diabetes. However, this approach is limited by an inadequate supply of cells with that property, which is exhibited only by pancreatic β-cells. The development of expanded populations of human β-cells that can be used for cell transplantation is therefore a major goal of diabetes research (D. R. W. Group, “Conquering diabetes: a strategic plan for the 21st century” NIH Publication No. 99-4398 (National Institutes of Health, 1999)). A number of alternative approaches are being pursued to achieve that goal, including using porcine tissue as a xenograft (Groth et al., J Mol Med 77:153-4 (1999)), expansion of primary human β-cells with growth factors and extracellular matrix (Beattie et al., Diabetes 48:1013-9 (1999)), and generation of immortalized cell lines that exhibit glucose-responsive insulin secretion (Levine, Diabetes/Metabolism Reviews 1: 209-46 (1997)).

Although there has been great interest in using porcine islets, they are difficult to manipulate in vitro and concerns have been raised about endogenous and exogenous xenobiotic viruses being transmitted to graft recipients (Weiss, Nature 391:327-8 (1998)). With primary human β-cells, entry into the cell cycle can be achieved using hepatocyte growth factor/scatter factor (“HGF/SF”) plus extracellular matrix (“ECM”) (Beattie et al., Diabetes 48:1013-9 (1999), Hayek et al., Diabetes 44:1458-1460 (1995)). However, this combination, while resulting in a 2-3×10⁴-fold expansion in the number of cells, is limited by cellular senescence and loss of differentiated function, particularly pancreatic hormone expression (Beattie et al., Diabetes 48:1013-9 (1999)).

Immortalized cell lines from the human endocrine pancreas have been created to develop β-cell lines that exhibit glucose responsive insulin secretion (Wang et al., Cell Transplantation 6:59-67 (1997), Wang et al., Transplantation Proceedings 29:2219 (1997), Halvorsen et al., Molecular and Cellular Biology 19:1864-1870 (1999)). The cell lines are made by infecting primary cultures of cells from various sources including adult islets, fetal islets, and purified β-cells, with viral vectors expressing the potent dominant oncogenes such as SV40 T antigen and H-ras^{val12} (Wang et al., Cell Transplantation 6:59-67 (1997), Wang et al., Transplantation Proceedings 29:2219 (1997), Halvorsen et al., Molecular and Cellular Biology 19:1864-1870 (1999); see also U.S. Pat. No. 5,723,333). The combined effect of those oncogenes is to trigger growth factor-independent and extracellular matrix (ECM)-independent entry into the cell cycle, as well as to prolong the life span of the cells from 10-15 population doublings or primary cells to approximately 150 doubling for the oncogene-expressing cells (Halvorsen et al., Molecular and Cellular Biology 19:1864-1870 (1999)). Further introduction of the gene encoding the hTRT component of telomerase results in immortalization, allowing the cells to be grown indefinitely (Halvorsen et al., Molecular and Cellular Biology 19:1864-1870 (1999)). Although the cell lines grow indefinitely, they lose differentiated function, similar to growth-stimulated primary β-cells.

Methods of stimulating differentiation of the cell lines into insulin-secreting β-cells and maintaining insulin secretion are therefore desired. Such cells could then be transplanted in vivo as a treatment for diabetes. The present invention addresses this and other problems.

### BRIEF SUMMARY OF THE INVENTION

The present invention provides methods for inducing insulin gene expression in cells. In some embodiments, the methods comprise the steps of: (i) providing a cell that expresses a PDX-1 polynucleotide; and (ii) contacting the cell with a histone deacetylase inhibitor, thereby inducing insulin gene expression in the cells. In some embodiments, the contacting step results in an induction of insulin expression at least two-fold compared to a cell not contacted by the histone deacetylase inhibitor.

In some embodiments, the cell further expresses a heterologous PDX-1 polynucleotide. In some embodiments, the PDX-1 polynucleotide hybridizes to a nucleotide sequence encoding SEQ ID NO:1 following at least one wash in 0.2×SSC at 55° C. for 20 minutes. In some embodiments, the PDX-1 polynucleotide encodes SEQ ID NO: 1.

In some embodiments, the cell expresses a NeuroD polynucleotide. In some embodiments, the cell expresses a heterologous NeuroD polynucleotide. In some embodiments, the NeuroD/BETA2 polynucleotide hybridizes to a nucleotide sequence encoding SEQ ID NO:2 following at least one wash in 0.2×SSC at 55° C. for 20 minutes. In some embodiments, the NeuroD/BETA2 polynucleotide encodes SEQ ID NO:2.

In some embodiments, the cell produces a detectable amount of insulin prior to contacting the cell with the histone deacetylase inhibitor.

In some embodiments, the inhibitor is selected from the group consisting of butyrates, hydroxamic acids, cyclic peptides and benzamides. In some embodiments, the inhibitor is selected from the group consisting of valproic acid, 4-phenylbutyrate, sodium butyrate, trichostatin A, suberoyl anilide hydroxamic acid (SAHA), oxamflatin, trapoxin B, FR901228, apicidin, chlamydocin, depuecin, scriptaid, depsipeptide, and N-acetyldinaline

In some embodiments, the methods further comprise contacting the cells with a GLP-1 receptor agonist. In some embodiments, the GLP-1 receptor agonist is a GLP-1 analog. In some embodiments, the GLP-1 receptor agonist has an amino acid sequence of a naturally-occurring peptide. In some embodiments, the GLP-1 receptor agonist is GLP-1, exendin-3, or exendin-4.

In some embodiments, the cell is a pancreatic β-cell. In some embodiments, the β-cells are human β-cells.

In some embodiments, the cells express a recombinant oncogene. In some embodiments, the cells express more than one recombinant oncogene. In some embodiments, the cells express a recombinant telomerase gene.

The invention also provides methods of identifying a compound that modulates β-cell function. In some embodiments, the methods comprise the steps of contacting a cell with a compound in the presence of a histone deactylase inhibitor, wherein the cell expresses a PDX-1 polynucleotide; and determining the effect of the compound on cell function. In some embodiments, β-cell function comprises insulin expression.

In some embodiments, insulin expression increases when the cell is contacted with the compound.

In some embodiments, the inhibitor is selected from the group consisting of butyrates, hydroxamic acids, cyclic peptides and benzamides. In some embodiments, the inhibitor is selected from the group consisting of valproic acid, 4-phenylbutyrate, sodium butyrate, trichostatin A, suberoyl anilide hydroxamic acid (SAHA), oxamflatin, trapoxin B, FR901228, apicidin, chlamydocin, depuecin, scriptaid, depsipeptide, and N-acetyldinaline.

In some embodiments, the β-cell expresses a NeuroD/BETA2 polynucleotide.

In some embodiments, the methods further comprise contacting the cells with a GLP-1 receptor agonist. In some embodiments, the GLP-1 receptor agonist is a GLP-1 analog. In some embodiments, the GLP-1 receptor agonist has an amino acid sequence of a naturally occurring peptide. In some embodiments, the GLP-1 receptor agonist is GLP-1, exendin-3, or exendin-4.

In some embodiments, the β-cell is a human cell.

The present invention also provides cultures of cells expressing PDX-1, wherein the culture comprises a histone deacetylase inhibitor. In some embodiments, the cells express a heterologous PDX-1 polynucleotide. In some embodiments, insulin expression of the cells is at least two-fold higher than cells in a culture lacking the histone deacetylase inhibitor.

In some embodiments, the cells further express a heterologous PDX-1 polynucleotide. In some embodiments, the PDX-1 polynucleotide hybridizes to a nucleotide sequence encoding SEQ ID NO: 1 following at least one wash in 0.2×SSC at 55° C. for 20 minutes. In some embodiments, the PDX-1 polynucleotide encodes SEQ ID NO:1.

In some embodiments, the cells express a NeuroD polynucleotide. In some embodiments, the cells express a heterologous NeuroD polynucleotide. In some embodiments, the NeuroD/BETA2 polynucleotide hybridizes to a nucleotide sequence encoding SEQ ID NO:2 following at least one wash in 0.2×SSC at 55° C. for 20 minutes. In some embodiments, the NeuroD/BETA2 polynucleotide encodes SEQ ID NO:2.

In some embodiments, the cells produce a detectable amount of insulin prior to contacting the cells with the histone deacetylase inhibitor.

In some embodiments, the inhibitor is selected from the group consisting of butyrates, hydroxamic acids, cyclic peptides and benzamides. In some embodiments, the inhibitor is selected from the group consisting of valproic acid, 4-phenylbutyrate, sodium butyrate, trichostatin A, suberoyl anilide hydroxamic acid (SAHA), oxamflatin, trapoxin B, FR901228, apicidin, chlamydocin, depuecin, scriptaid, depsipeptide, and N-acetyldinaline.

In some embodiments, the culture further comprises a GLP-1 receptor agonist. In some embodiments, the GLP-1 receptor agonist is a GLP-1 analog. In some embodiments, the GLP-1 receptor agonist has an amino acid sequence of a naturally occurring peptide. In some embodiments, the GLP-1 receptor agonist is GLP-1, exendin-3, or exendin-4.

In some embodiments, the cells are pancreatic β-cells. In some embodiments, the β-cells are human β-cells.

In some embodiments, the cells express a recombinant oncogene. In some embodiments, the cells express more than one recombinant oncogene. In some embodiments, the cells express a recombinant telomerase gene.

### Definitions

As used herein, the following terms have the meanings ascribed to them unless specified otherwise.

“Histone deacetylase” refers to enzymes that remove acetyl groups from histones. See, e.g., Kochbin, S. et al., Curr. Opin. Genet. Dev. 11:162-166 (2001); Gray et al, Exp. Cell Res. 262:75-83 (2001). Histone deacetylases counteract the effect of acetyltransferases and can act as gene repressors by condensing chromatin. Human histone deacetylases belong to at least three classes of proteins based on their homology to yeast proteins. One class of human histone deacetylases are homologous to yeast RPD3 and are designated HDAC 1, 2, 3, and 8. Class II histone deacetylases have homology to yeast HDA1 and include, e.g., HDAC 4, 5, 6, and 7. Class III histone deacetylases have NAD⁺ dependent activity and have homology to yeast and mouse silent information regulatory 2.

A “histone deacetylase inhibitor” refers to a molecule that inhibits the activity of histone deacetylase. A number of histone deacetylase inhibitors have been described in the art. See, e.g., Marks, et al, Curr. Opin. Oncol. 13(6):477-83 (2001); Jung, Curr. Med. Chem. 8(12):1505-1511 (2001). Exemplary histone deacetylase inhibitors include, e.g., short chain fatty acids (e.g., butyrates (such as 4-phenylbutyrate and sodium butyrate), hydroxamic acids (e.g., trichostatin A, suberoyl anilide hydroxamic acid (SAHA), oxamflatin, and CHAP compounds), cyclic tetrapeptides containing a 2-amino-8-oxo-9,10-epoxy-decanoyl moiety (e.g., trapoxin B), cyclic peptides (e.g., FR901228 and apicidin) and benzamides (e.g., MS-275), as well as TPX-HA analogs, chlamydocin, depuecin, scriptaid, depsipeptide, and N-acetyldinaline.

“Inducing insulin gene expression” refers to increasing, in a cell or culture of cells, the level of expression from the insulin gene by at least about 10%, preferably at least about 25% or 50% more than a negative control culture (e.g., a cell not contacted with a histone deacetylase inhibitor). Induction can be as much as at least about 2-, 3-, 4-, 5-, 8-, 10-, 20-, 50-, 100-fold or more compared to a negative control culture (e.g., a cell not contacted with a histone deacetylase inhibitor). Insulin gene expression can be measured by methods known to those of skill in the art, e.g., by measuring insulin RNA expression, preproinsulin, proinsulin, insulin, or c-peptide production, e.g., using PCR, hybridization, and immunoassays.

Cells that “secrete insulin in response to glucose” are cells or a cell culture that, in comparison to a negative control (either non-insulin responsive cells or insulin responsive cells that are not exposed to glucose), have increased insulin secretion in response to glucose of at least about 10%, preferably 25%, 50%, 100%, 500%, 1000%, 5000%, or higher than the control cells (measured as described above).

“Endocrine pancreas cells” refers to cells originally derived from an adult or fetal pancreas, such as islet cells. “Cultured” endocrine pancreas cells refers to primary cultures as well as cells that have been transformed with genes such as an oncogene, e.g., SV40 T antigen, ras, or a telomerase gene (e.g., hTRT).

A “GLP-1 receptor agonist” refers to GLP-1, a GLP-1 analog, or a naturally occurring peptide that binds to the GLP-1 receptor (e.g., exendin-3 or -4), thereby activating signal transduction from the receptor.

“Culturing” refers to growing cells ex vivo or in vitro. Cultured cells can be non-naturally occurring cells, e.g., cells that have been transduced with an exogenous gene such as an oncogene or a transcription factor such as NeuroD/BETA2 and/or PDX-1. Cultured cells can also be naturally occurring isolates or primary cultures.

A “stable” cell line or culture is one that can grow in vitro for an extended period of time, such as for at least about 50 cell divisions, or for about 6 months, more preferably for at least about 150 cell divisions, or at least about ten months, and more preferably at least about a year.

“Modulating β-cell function” refers to a compound that increases (activates) or decreases (inhibits) glucose responsive insulin secretion of an endocrine pancreas cell. Glucose responsive insulin secretion can be measured by a number of methods, including analysis of insulin mRNA expression, preproinsulin production, proinsulin production, insulin production, and c-peptide production, using standard methods known to of skill in the art. To examine the extent of modulation, cultured cells are treated with a potential activator or inhibitor and are compared to control samples without the activator or inhibitor. Control samples (untreated with inhibitors or activators or not in cell-to-cell contact or not contacted with a histone deacetylase inhibitor) are assigned a relative insulin value of 100%. Inhibition is achieved when the insulin value relative to the control is about 90%, preferably 75%, 50%, and more preferably 25-0%. Activation is achieved when the insulin value relative to the control is 110%, more preferably 125%, 150%, and most preferably at least 200-500% higher or 1000% or higher.

A “diabetic subject” is a mammalian subject, often a human subject, that has any type of diabetes, including primary and secondary diabetes, type 1 NIDDM-transient, type 1 IDDM, type 2 IDDM-transient, type 2 NIDDM, and type 2 MODY, as described in Harrison 's Internal Medicine, 14th ed. 1998.

“Expressing” a gene refers to expression of a recombinant or endogenous gene, e.g., resulting in mRNA or protein production from the gene. A recombinant gene can be integrated into the genome or in an extrachromosomal element.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

The term “immunoassay” is an assay that uses an antibody to specifically bind an antigen, e.g., ELISA, western blot, RIA, immunoprecipitation and the like. The immunoassay is characterized by the use of specific binding properties of a particular antibody to isolate, target, and/or quantify the antigen.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As for amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

(see, e.g., Creighton, Proteins (1984)).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter. In one embodiment of the invention the expression vector is a viral vector, preferably one that integrates into the host cell genome, such as a retroviral vector, or an adeno-associated viral vector. Examples of retroviruses, from which viral vectors of the invention can be derived, include avian retroviruses such as avian erythroblastosis virus (AMV), avian leukosis virus (ALV), avian myeloblastosis virus (ABV), avian sarcoma virus (ACV), spleen necrosis virus (SNV), and Rous sarcoma virus (RSV); non-avian retroviruses such as bovine leukemia virus (BLV); feline retroviruses such as feline leukemia virus (FeLV) or feline sarcoma virus (FeSV); murine retroviruses such as murine leukemia virus (MuLV), mouse mammary tumor virus (MMTV), murine sarcoma virus (MSV), and Moloney murine sarcoma virus (MoMSV); rat sarcoma virus (RaSV); and primate retroviruses such as human T-cell lymphotropic viruses 1 and 2 (HTLV-1, 2) and simian sarcoma virus (SSV). Many other suitable retroviruses are know to those of skill in the art. Often the viruses are replication deficient, i.e., capable of integration into the host genome but not capable of replication to provide infective virus. In another embodiment of the invention, the vector is a transient vector such as an adenoviral vector, e.g., for transducing the cells with a recombinase to delete the integrated oncogenes.

A “PDX-1 polynucleotide” refers to a polynucleotide encoding a polypeptide substantially identical to SEQ ID NO: 1. Exemplary PDX-1 polynucleotides are described in, e.g., Sander et al, J. Mol. Med. 71:327-340 (1997).

A “NeuroD/BETA2 polynucleotide” refers to a polynucleotide encoding a polypeptide substantially identical to SEQ ID NO:2. Exemplary NeuroD/BETA2 polynucleotide are described in, e.g., U.S. Pat. No. 5,795,723; Miyachi, T., et al. Mol. Brain Res. 69, 223-231 (1999); Lee, et al Science 268:836-844 (1995); Wilson et al., Nature 368, 32-38 (1994); and Naya et al., Genes Dev. 9:1009-1019 (1995)).

The terms “identical” or percent “identity,” in the context of two or more nucleic acids or polypeptide sequences, refer to two or more sequences or subsequences that are the same. “Substantially identical” refers to two or more nucleic acids or polypeptide sequences having a specified percentage of amino acid residues or nucleotides that are the same (i.e., at least 60% identity, optionally 65%, 70%, 75%, 80%, 85%, 90%, or 95% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity or substantial identity exists over a region that is at least about 50 nucleotides in length, or more preferably over a region that is 100 to 500 or 1000 or more nucleotides or amino acids in length.

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or 5×SSC, 1% SDS, incubating at 65° C., with wash in 0.2×SSC at 55° C., 60° C., or 65° C. (and optionally 0.1% SDS). Such washes can be performed for 5, 15, 30, 60, 120, or more minutes.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. Such washes can be performed for 5, 15, 30, 60, 120, or more minutes. A positive hybridization is at least twice background. Those of ordinary skill will readily recognize that alternative hybridization and wash conditions can be utilized to provide conditions of similar stringency.

### BRIEF DESCRIPTION OF THE DRAWINGS

FIG. 1 illustrates induction of insulin expression resulting from treatment with trichostatin A (TSA). Insulin mRNA was analyzed by RT-PCR. RT-PCR for the housekeeping gene porphobilinogen deaminase was performed to ensure that equal amounts of cDNA were used. The experiment has been repeated three times and the figure shown here is representative.

FIG. 2 is a bar graph illustrating expression of an insulin promoter in HeLa cells transformed with PDX-1. The graph illustrates differences in expression from the insulin promoter in the presence or absence of the histone deacetylase inhibitor TSA. Insulin promoter activity was determined based on expression of chloramphenicol acetyl transferase (CAT) reporter activity.

### DETAILED DESCRIPTION OF THE INVENTION

I. Introduction

The present invention provides methods and compositions for inducing insulin expression in cells. As described herein, it has been discovered that contacting cells committed to a β-cell lineage with a histone deacetylase significantly induces expression of insulin. Inducing insulin expression in cells has many uses, including, e.g., supplementing insulin production of diabetic patients.

II. Cells of the Invention

The present invention provides methods and compositions for inducing insulin expression in cells. Any cell committed to a β-cell lineage can be used according to the methods described herein. In some embodiments, the cells will express a detectable amount of insulin before the cells are contacted with a histone deacetylase inhibitor.

Insulin expression can be induced in cells committed to a β-cell lineage by contacting the cells with a histone deacetylase inhibitor. Concentrations of the inhibitor can vary depending on the exact conditions, cells and inhibitor used. In some aspects, the inhibitor concentration is from about 1 nM to 100 μM, and often is about between 1-50 μM.

Typically, the inhibitor is contacted with the cells for a period of time. For example, the inhibitor is typically contacted to the cell for at least about one hour and more typically is contacted for at least 12, 24, 48 or more hours.

Cells committed to a β-cell lineage can often be recognized by testing the cells for expression of β-cell specific gene expression. β-cell specific genes include, e.g., PDX-1. PDX-1 is involved in the regulation of insulin expression. See, e.g., PCT Application No. 01/07628. Therefore, cells expressing PDX-1 are likely committed to β-cell differentiation.

The cells of the invention can express either endogenous or recombinant PDX-1 having PDX-1 activity, e.g., alleles, polymorphic variants, and orthologs (see, e.g., Sander et al., J. Mol. Med. 71:327-340 (1997)). Endogenous expression of PDX-1 can be induced using transcription factors such as hepatocyte nuclear factor 3 beta, which is involved in pancreatic β-cell expression of the PDX-1 gene (see, e.g., Wu et al., Molecular and Cellular Biology 17:6002-6013 (1997)). Recombinant PDX-1 is delivered to the cells using expression vectors, e.g., viral vectors such as retroviral vectors, as described above.

Other exemplary β-cell specific genes include, e.g., NKX6.1 (e.g., Sander et al., Development 127:5533-5540 (2000)) and PAX4 (e.g., Sosa-Pineda, et al., Nature 386:399-402 (1997)). These gene products are typically expressed in β-cells. Other relevant endocrine pancreas gene markers, though not necessarily β-cell specific markers, include, e.g., PAX6 (see, e.g., Larsson, et al., Mechanisms of Development 79:153-159 (1998)), NKX2.2 (M. Sander, et al., Development 127:5533-5540 (2000)), sulfonylurea receptor (see, e.g., Aguilar-Bryan et al., Science 268:423-426 (1995)), the GLP-1 receptor (see, e.g., Salapatek et al., Mol Endocrinol 13(8):1305-17 (1999)) and glucokinase (see, e.g., Matschinsky et al., Diabetes 47(3):307-15 (1998).

In one embodiment, cells used in the practice of the invention express one or more oncogenes, such as SV40 T antigen and Hras^{val12}, which minimally transform the cells but stimulate growth and bypass cellular senescence. Other suitable oncogenes include, e.g., HPV E7, HPV E6, c-myc, and CDK4 (see also U.S. Pat. No. 5,723,333). In addition, the cells can be transduced with an oncogene encoding mammalian telomerase, such as hTRT, to facilitate immortalization. Suitable oncogenes can be identified by those of skill in the art, and partial lists of oncogenes are provided in Bishop et al., RNA Tumor Viruses, vol. 1, pp. 1004-1005 (Weiss et al., eds, 1984), and Watson et al., Molecular Biology of the Gene (4^{th} ed. 1987). In some cases the oncogenes provide growth factor-independent and ECM-independent entry into the cell cycle. Often the oncogenes are dominant oncogenes. In some embodiments, the oncogenes are delivered to the cells using a viral vector, preferably a retroviral vector, although any suitable expression vector can be used to transduce the cells (see, e.g., U.S. Pat. No. 5,723,333, which describes construction of vectors encoding one or more oncogenes and transduction of pancreas endocrine cells, see also Halvorsen et al., Molecular and Cellular Biology 19:1864-1870 (1999)).

The vector used to create the cell lines can incorporate recombinase sites, such as lox sites, so that the oncogenes can be deleted by expression of a recombinase, such as the cre recombinase, in the cells following expansion (Halvorsen et al., Molecular and Cellular Biology 19:1864-1870 (1999)). Deletion of the oncogenes is useful for cells that are to be transplanted in to a mammalian subject. Other recombinase systems include Saccharomyces cerevisiae FLP/FRT, lambda att/Int, R recombinase of Zygosaccharomyces rouxii. In addition, transposable elements and transposases could be used. Deletion of the oncogene can be confirmed, e.g., by analysis of oncogene RNA or protein expression, or by Southern blot analysis.

The cultured cells of the invention can express either endogenous or recombinant NeuroD/BETA2 including, e.g., alleles, polymorphic variants, and orthologs having NeuroD/BETA2 activity (see, e.g., U.S. Pat. No. 5,795,723; Miyachi, T., et al. Mol. Brain Res. 69, 223-231 (1999); Lee, et al. Science 268:836-844 (1995); Wilson et al., Nature 368, 32-38 (1994); Naya et al., Genes Dev. 9:1009-1019 (1995)). Human NeuroD/BETA2 alleles and variants are particularly desirable. Recombinant PDX-1 is delivered to the cells using expression vectors, e.g., viral vectors such as retroviral vectors, as described above.

The vectors used to transduce the cells can be any suitable vector, including viral vectors such as retroviral vectors. Preferably, the vector is one that provides stable transformation of the cells, as opposed to transient transformation.

In some aspects, GLP-1 receptor agonists are also administered to the cells of the invention. GLP-1 receptor antagonists include naturally occurring peptides such as GLP-1, exendin-3, and exendin-4 (see, e.g., U.S. Pat. No. 5,424,286; U.S. Pat. No. 5,705,483, U.S. Pat. No. 5,977,071; U.S. Pat. No. 5,670,360; U.S. Pat. No. 5,614,492), GLP-1 analogs (see, e.g., U.S. Pat. No. 5,545,618 and U.S. Pat. No. 5,981,488), and small molecule analogs. GLP-1 receptor agonists may be tested for activity as described in U.S. Pat. No. 5,981,488. Cells are contacted with a GLP-1 receptor agonist in a time and amount effective to induce insulin mRNA expression. See, e.g., PCT Application No. 01/07628. Typically, the cells are contacted with the GLP-1 receptor agonists for a discrete time period, as the GLP-1 receptor agonist can act as a switch for insulin gene expression. Continuous administration of the GLP-1 receptor agonist is therefore not required.

This invention relies upon routine techniques in the field of cell culture, and suitable methods can be determined by those of skill in the art using known methodology (see, e.g., Freshney et al., Culture of Animal Cells (3^{rd} ed. 1994)). In general, the cell culture environment includes consideration of such factors as the substrate for cell growth, cell density and cell contract, the gas phase, the medium, and temperature.

For the cells of the invention that are cultured under adherent conditions, plastic dishes, flasks, roller bottles, or microcarriers in suspension are used. Other artificial substrates can be used such as glass and metals. The substrate is often treated by etching, or by coating with substances such as collagen, chondronectin, fibronectin, and laminin. The type of culture vessel depends on the culture conditions, e.g., multi-well plates, petri dishes, tissue culture tubes, flasks, roller bottles, and the like.

Cells are grown at optimal densities that are determined empirically based on the cell type. Cells are passaged when the cell density is above optimal.

Cultured cells are normally grown in an incubator that provides a suitable temperature, e.g., the body temperature of the animal from which is the cells were obtained, accounting for regional variations in temperature. Generally, 37° C. is the preferred temperature for cell culture. Most incubators are humidified to approximately atmospheric conditions.

Important constituents of the gas phase are oxygen and carbon dioxide. Typically, atmospheric oxygen tensions are used for cell cultures. Culture vessels are usually vented into the incubator atmosphere to allow gas exchange by using gas permeable caps or by preventing sealing of the culture vessels. Carbon dioxide plays a role in pH stabilization, along with buffer in the cell media and is typically present at a concentration of 1-10% in the incubator. The preferred CO₂ concentration typically is 5%.

Defined cell media are available as packaged, premixed powders or presterilized solutions. Examples of commonly used media include DME, RPMI 1640, DMEM, Iscove's complete media, or McCoy's Medium (see, e.g., GibcoBRL/Life Technologies Catalogue and Reference Guide; Sigma Catalogue). Typically, low glucose DME or RPMI 1640 are used in the methods of the invention. Defined cell culture media are often supplemented with 5-20% serum, typically heat inactivated, e.g., human horse, calf, and fetal bovine serum. Typically, 10% fetal bovine serum is used in the methods of the invention. The culture medium is usually buffered to maintain the cells at a pH preferably from 7.2-7.4. Other supplements to the media include, e.g., antibiotics, amino acids, sugars, and growth factors such as hepatocyte growth factor/scatter factor.

In some aspects, cells committed to a β-cell lineage are extracted from a human and subsequently contacted with a histone deacetylase inhibitor. The embodiments are useful for treating diabetic subjects by implanting cells that express insulin in a glucose-dependent manner. Cells can be extracted from the subject to be treated (thereby avoiding immune-based rejection of the implant) or can be from a second individual.

Methods of isolating pancreatic islet cells are known in the art. See, e.g., Field et al., Transplantation 61:1554 (1996); Linetsky et al., Diabetes 46:1120 (1997). Fresh pancreatic tissue can be divided by mincing, teasing, comminution and/or collagenase digestion. The islets are then isolated from contaminating cells and materials by washing, filtering, centrifuging or picking procedures. Methods and apparatus for isolating and purifying islet cells are described in, e.g., U.S. Pat. Nos. 5,447,863, 5,322,790, 5,273,904, and 4,868,121. The isolated pancreatic cells may optionally be cultured prior to microencapsulation, using any suitable method of culturing islet cells as is known in the art. See, e.g., U.S. Pat. No. 5,821,121. Isolated cells may be cultured in a medium under conditions that helps to eliminate antigenic components. See, e.g., Transplant. Proc. 14:714-23 (1982)).

Cells produced according to the present invention may be transplanted into subjects as a treatment for insulin-dependent diabetes; such transplantation may be into the peritoneal cavity of the subject. An amount of cells to produce sufficient insulin to control glycemia in the subject is provided by any suitable means, including but not limited to surgical implantation and intraperitoneal injection. Where the cells are islet cells, the International Islet Transplant Registry has recommended transplants of at least 6,000 islets, equivalent to 150 μm in size, per kilogram of recipient body weight, to achieve euglycemia. However, it will be apparent to those skilled in the art that the quantity of cells transplanted depends on the ability of the cells to provide insulin in vivo, in response to glucose stimulation.

To reduce immunorejection by the transplant patient, the cells may additionally contain genes which reduces immunogenicity in the genetically modified cell lines. An example of such a gene is the adenoviral P19 gene that encodes a transmembrane glycoprotein (gp19K). gp19K is localized in the endoplasmic reticulum and binds to class I antigen (Ag) of the major histocompatibility complex (MHC). This binding blocks the transport of class I Ag to the surface of the infected cell and prevents class-I-restricted cytolysis by cytotoxic T lymphocyte (CTL) (Paabo, S., et al., Cell, 50:311-317 (1987); Wold, W. S. M., and Gooding, L. R., Mol. Biol. Med., 6:433-452 (1989)).

Alternatively, to further reduce host versus graft immune rejection, one may use the patient's cells and coaxed their growth by exposing them to mitotic agents, such as collagenase, dexamethasone, fibroblast growth factor, before or after contacting the cells with a histone deacetylase inhibitor.

Besides transplantation, the genetically modified cell lines can be cultured and used to produce the desired gene products in vitro that are harvested and purified according to methods known in the art.

The cell lines described herein also provide well characterized cells for other purposes such as for screening of chemicals which interact with proteins on the cells' surface, e.g., for therapeutic uses.

III. Pharmaceutical Compositions and Administration

Pharmaceutically acceptable carriers are determined in part by the particular composition being administered (e.g., a cell or small molecule), as well as by the particular method used to administer the composition. Accordingly, there are a wide variety of suitable formulations of pharmaceutical compositions of the present invention (see, e.g., Remington's Pharmaceutical Sciences, 17th ed., 1989).

Formulations suitable for parenteral administration, such as, for example, by intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by direct surgical transplantation under the kidney, intraportal administration, intravenous infusion, or intraperitoneal infusion.

Injection solutions and suspensions can be prepared from sterile powders, granules, and tablets. The dose administered to a patient, in the context of the present invention should be sufficient to effect a beneficial therapeutic response in the patient over time. The dose will be determined by the efficacy of the particular cells employed and the condition of the patient, as well as the body weight or surface area of the patient to be treated. The size of the dose also will be determined by the existence, nature, and extent of any adverse side-effects that accompany the administration of a particular vector, or transduced cell type in a particular patient.

In determining the effective amount of the cells to be administered in the treatment or prophylaxis of conditions owing to diminished or aberrant insulin expression, the physician evaluates cell toxicity, transplantation reactions, progression of the disease, and the production of anti-cell antibodies. For administration, cells of the present invention can be administered in an amount effective to provide normalized glucose responsive-insulin production and normalized glucose levels to the subject, taking into account the side-effects of the cell type at various concentrations, as applied to the mass and overall health of the patient. Administration can be accomplished via single or divided doses.

IV. Assays For Modulators of β-Cell Function

A. Assays

Assays using the cells of the invention (e.g., in the presence of a histone deacetylase inhibitor) can be used to test for inhibitors and activators of β-cell function, e.g., insulin production and/or glucose responsive insulin production. Such modulators are useful for treating various disorders involving glucose metabolism, such as diabetes and hypoglycemia. Treatment of dysfunctions include, e.g., diabetes mellitus (all types); hyperinsulinism caused by insulinoma, drug-related, e.g., sulfonylureas or excessive insulin, immune disease with insulin or insulin receptor antibodies, etc. (see, e.g., Harrison's Internal Medicine (14^{th} ed. 1998)).

Modulation is tested using the cultures of the invention by measuring insulin gene expression, optionally with administration of glucose, e.g., analysis of insulin mRNA expression using northern blot, dot blot, PCR, oligonucleotide arrays, and the like; and analysis of insulin protein expression (preproinsulin, proinsulin, insulin, or c-peptide) using, e.g., western blots, radio immune assays, ELISAs, and the like. Downstream effects of insulin modulation can also be examined. Physical or chemical changes can be measured to determine the functional effect of the compound on β cell function. Samples or assays that are treated with a potential inhibitor or activator are compared to control samples without the test compound, to examine the extent of modulation.

B. Modulators

The compounds tested as modulators of β-cell function can be any small chemical compound, or a macromolecule, such as a protein, sugar, nucleic acid or lipid. Typically, test compounds will be small chemical molecules and peptides. Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In some embodiments, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

The assays can be solid phase or solution phase assays. In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 96 modulators. If 1536 well plates are used, then a single plate can easily assay from about 100-about 1500 different compounds. It is possible to assay many plates per day; assay screens for up to about 6,000, 20,000, 50,000, or 100,000 or more different compounds is possible using the integrated systems of the invention.

V. General Molecular Biology Methods

This invention relies on routine techniques in the field of recombinant genetics. Basic texts disclosing the general methods of use in this invention include Sambrook et al., Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et. al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

The sequence of the cloned genes and synthetic oligonucleotides can be verified after cloning using, e.g., the chain termination method for sequencing double-stranded templates of Wallace et al., Gene 16:21-26 (1981).

In general, the nucleic acids encoding the subject proteins are cloned from DNA sequence libraries that are made to encode cDNA or genomic DNA. The particular sequences can be located by hybridizing with an oligonucleotide probe, the sequence of which can be derived from the sequences disclosed herein, which provide a reference for PCR primers and defines suitable regions for isolating specific probes (e.g., for a β-cell specific gene or gene product). Alternatively, where the sequence is cloned into an expression library, the expressed recombinant protein can be detected immunologically with antisera or purified antibodies made against a polypeptide of interest, including those disclosed herein.

Methods for making and screening genomic and cDNA libraries are well known to those of skill in the art (see, e.g., Gubler and Hoffman Gene 25:263-269 (1983); Benton and Davis Science, 196:180-182 (1977); and Sambrook, supra). Briefly, to make the cDNA library, one should choose a source that is rich in mRNA. The mRNA can then be made into cDNA, ligated into a recombinant vector, and transfected into a recombinant host for propagation, screening and cloning. For a genomic library, the DNA is extracted from a suitable tissue and either mechanically sheared or enzymatically digested to yield fragments of preferably about 5-100 kb. The fragments are then separated by gradient centrifugation from undesired sizes and are constructed in bacteriophage lambda vectors. These vectors and phage are packaged in vitro, and the recombinant phages are analyzed by plaque hybridization. Colony hybridization is carried out as generally described in Grunstein et al., Proc. Natl. Acad. Sci. USA., 72:3961-3965 (1975).

An alternative method combines the use of synthetic oligonucleotide primers with polymerase extension on an mRNA or DNA template. Suitable primers can be designed from sequences disclosed herein. This polymerase chain reaction (PCR) method amplifies the nucleic acids encoding the protein of interest directly from mRNA, cDNA, genomic libraries or cDNA libraries. Restriction endonuclease sites can be incorporated into the primers. Polymerase chain reaction or other in vitro amplification methods may also be useful, for example, to clone nucleic acids encoding specific proteins and express said proteins, to synthesize nucleic acids that will be used as probes for detecting the presence of mRNA encoding a polypeptide of the invention in physiological samples, for nucleic acid sequencing, or for other purposes (see, U.S. Pat. Nos. 4,683,195 and 4,683,202). Genes amplified by a PCR reaction can be purified from agarose gels and cloned into an appropriate vector.

Appropriate primers and probes for identifying the genes encoding a polypeptide of the invention from mammalian tissues can be derived from the sequences provided herein. For a general overview of PCR, see, Innis et al. PCR Protocols: A Guide to Methods and Applications, Academic Press, San Diego (1990).

Synthetic oligonucleotides can be used to construct genes. This is done using a series of overlapping oligonucleotides, usually 40-120 bp in length, representing both the sense and anti-sense strands of the gene. These DNA fragments are then annealed, ligated and cloned.

All publications and patent applications cited in this specification are herein incorporated by reference as if each individual publication or patent application were specifically and individually indicated to be incorporated by reference.

The following examples are provided by way of illustration only and not by way of limitation. Those of skill in the art will readily recognize a variety of noncritical parameters that could be changed or modified to yield essentially similar results.

### EXAMPLE

It is known that cell-to-cell contact can greatly increase insulin expression in cultured cells. See, PCT Application No.01/07628. The data presented herein demonstrates that cell-to-cell contact can be supplanted by treating cultured cells with a histone deacetylase inhibitor.

Treatment of the human pancreatic endocrine cell line, TRM-6/PDX-1, with the histone deacetylase inhibitor, TSA, bypassed the need for cell-cell contact to achieve high levels of somatostatin gene expression. The histone deacetylase inhibitor trichostatin A also induced insulin gene expression in TRM-6 cells.

Somatastatin Production

Treatment of TRM-6 cells expressing PDX-1 with the histone deacetylase inhibitor trichostatin A (TSA) induced somatastatin mRNA expression in monolayer cultures. The cells were incubated with the inhibitor for approximately 24 hours and then expression of somatastatin expression was measured with RT-PCR. As little as 66 nM TSA lead to an increase in somatastatin induction, with a maximal induction at 6.6. μM of TSA.

Insulin Production

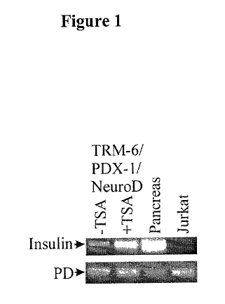

Monolayer cultures of TRM-6/PDX-1 also expressing the transcription factor NeuroD 1 (BETA2) express low levels of insulin gene expression. Treatment with 3.3 μM TSA for 24 hours greatly increases insulin expression in TRM-6/PDX-1/NeuroD1 cells (FIG. 1). These results provide important new information on the role of histone acetylation in the regulation of insulin gene expression.

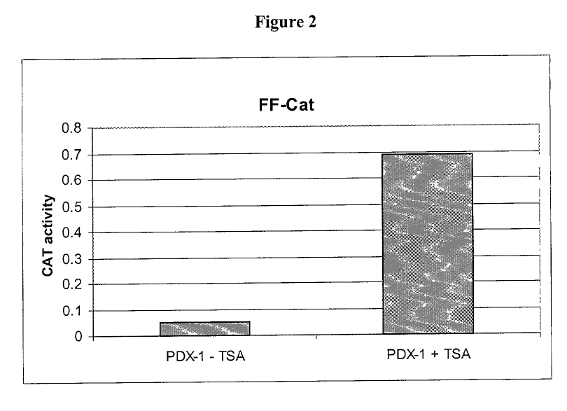

In addition, expression of an insulin promoter in HeLa cells was tested for the effect of the histone deacetylase inhibitor TSA on insulin expression. HeLa cells were transformed with PDX-1 and a plasmid comprising a portion of the insulin promoter operably linked to the chloramphenicol acetyl transferase (CAT) reporter gene. The effect of TSA and PDX-1 on insulin expression was then tested. As FIG. 2 illustrates, cells contacted with TSA had approximately 14 times the expression of cells not contacted with TSA.

What is claimed is:

1. A method for inducing insulin gene expression in cells, the method comprising the steps of:
(i) providing a cell that expresses a PDX-1 polynucleotide; and
(ii) contacting the cell with a histone deacetylase inhibitor, thereby inducing insulin gene expression in the cells.

2. The method of claim 1, wherein the contacting step results in an induction of insulin expression at least two-fold compared to a cell not contacted by the histone deacetylase inhibitor.

3. The method of claim 1, wherein the cell further expresses a heterologous PDX-1 polynucleotide.

4. The method of claim 1, wherein the cell expresses a NeuroD polynucleotide.

5. The method of claim 4, wherein the cell expresses a heterologous NeuroD polynucelotide.

6. The method of claim 1, wherein the cell is a pancreatic β-cell.

7. The method of claim 6, wherein the β-cells are human β-cells.

8. The method of claim 1, wherein the cell produces a detectable amount of insulin prior to contacting the cell with the histone deacetylase inhibitor.

9. The method of claim 1, wherein the inhibitor is selected from the group consisting of butyrates, hydroxamic acids, cyclic peptides and benzamides.

10. The method of claim 1, wherein the inhibitor is selected from the group consisting of valproic acid, 4-phenylbutyrate, sodium butyrate, trichostatin A, suberoyl anilide hydroxamic acid (SAHA), oxamflatin, trapoxin B, FR901228, apicidin, chlamydocin, depuecin, scriptaid, depsipeptide, and N-acetyldinaline

11. The method of claim 1, further comprising contacting the cells with a GLP-1 receptor agonist.

12. The method of claim 11, wherein the GLP-1 receptor agonist is a GLP-1 analog.

13. The method of claim 11, wherein the GLP-1 receptor agonist has an amino acid sequence of a naturally occurring peptide.

14. The method of claim 13, wherein the GLP-1 receptor agonist is GLP-1, exendin-3, or exendin-4.

15. The method of claim 1, wherein the cells express a recombinant oncogene.

16. The method of claim 15, wherein the cells express more than one recombinant oncogene.

17. The method of claim 1, wherein the cells express a recombinant telomerase gene.

18. A method of identifying a compound that modulates β-cell function, the method comprising the steps of contacting a cell with a compound in the presence of a histone deactylase inhibitor, wherein the cell expresses a PDX-1 polynucleotide; and
determining the effect of the compound on β-cell function.

19. The method of claim 18, wherein β-cell function comprises insulin expression.

20. The method of claim 18, wherein insulin expression increases when the cell is contacted with the compound.

21. The method of claim 18, wherein the inhibitor is selected from the group consisting of butyrates, hydroxamic acids, cyclic peptides and benzamides.

22. The method of claim 18, wherein the inhibitor is selected from the group consisting of valproic acid, 4-phenylbutyrate, sodium butyrate, trichostatin A, suberoyl anilide hydroxamic acid (SAHA), oxamflatin, trapoxin B, FR901228, apicidin, chlamydocin, depuecin, scriptaid, depsipeptide, and N-acetyldinaline

23. The method of claim 18, wherein the β-cell expresses a NeuroD/BETA2 polynucleotide.

24. The method of claim 18, further comprising contacting the β-cell with a GLP-1 receptor agonist.

25. The method of claim 24, wherein the GLP-1 receptor agonist is a GLP-1 analog.

26. The method of claim 24, wherein the GLP-1 receptor agonist has an amino acid sequence of a naturally occurring peptide.

27. The method of claim 26, wherein the GLP-1 receptor agonist is GLP-1, exendin-3, or exendin-4.

28. The method of claim 18, wherein the β-cell is a human cell.

29. A culture of cells expressing PDX-1, wherein the culture comprises a histone deacetylase inhibitor.

30. The culture of claim 29, wherein the cells express a heterologous PDX-1 polynucleotide.

31. The culture of claim 29, wherein insulin expression of the cells is at least two-fold higher than cells in a culture lacking the histone deacetylase inhibitor.

32. The culture of claim 29, wherein the inhibitor is selected from the group consisting of butyrates, hydroxamic acids, cyclic peptides and benzamides.

33. The culture of claim 29, wherein the inhibitor is selected from the group consisting of valproic acid, 4-phenylbutyrate, sodium butyrate, trichostatin A, suberoyl anilide hydroxamic acid (SAHA), oxamflatin, trapoxin B, FR901228, apicidin, chlamydocin, depuecin, scriptaid, depsipeptide, and N-acetyldinaline

34. The culture of claim 29, wherein the cell expresses a NeuroD polynucleotide.

35. The culture of claim 34, wherein the cell expresses a heterologous NeuroD polynucelotide.

36. The culture of claim 29, further comprising a GLP-1 receptor agonist.

37. The culture of claim 36, wherein the GLP-1 receptor agonist is a GLP-1 analog.

38. The culture of claim 36, wherein the GLP-1 receptor agonist has an amino acid sequence of a naturally occurring peptide.

39. The culture of claim 38, wherein the GLP-1 receptor agonist is GLP-1, exendin-3, or exendin-4.

40. The culture of claim 29, wherein the cells are β-cells.

41. The culture of claim 40, wherein the β-cells are human β-cells.

42. The culture of claim 29, wherein the β-cells express a recombinant oncogene.

43. The culture of claim 42, wherein the β-cells express more than one recombinant oncogene.

44. The culture of claim 40, wherein the β-cells express a recombinant telomerase gene.


SEQUENCE LISTING (2 sequences)

SEQ ID NO: 1 - protein, 283 aa, Homo sapiens
  human pancreas/duodenum homeobox-1 (PDX-1)
MNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPR

SEQ ID NO: 2 - protein, 356 aa, Homo sapiens
  human neurogenic differentiation factor 1 (NeuroD/BETA2)
MTKSYSESGLMGEPQPQGPPSWTDECLSSQDEEHEADKKEDDLEAMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD